(19) United States
(12) Patent Application Publication
(10) Pub. No.: US 2015/0290293 A1
(43) Pub. Date: Oct. 15, 2015

(54) METHODS FOR CHRONIC PAIN MANAGEMENT AND TREATMENT USING HCG

(71) Applicant: Neuralight HD, LLC, Phoenix, AZ (US)

(72) Inventors: Edson Conrad Hicks, JR., Phoenix, AZ (US); Constance T. Dutton, San Diego, CA (US)

(21) Appl. No.: 14/539,940

(22) Filed: Nov. 12, 2014

Related U.S. Application Data
(63) Continuation-in-part of application No. 14/218,448, filed on Mar. 18, 2014.
Application No. 14/218,448 is a continuation of application No. 13/633,739, filed on Oct. 2, 2012, now Pat. No. 8,680,088.
Application No. 13/633,739 is a continuation of application No. 13/311,250, filed on Dec. 5, 2011, now abandoned.
Application No. 13/311,250 is a continuation-in-part of application No. 13/211,101, filed on Aug. 16, 2011, now Pat. No. 8,680,086.
(60) Provisional application No. 61/475,908, filed on Apr. 15, 2011.

(51) Int. Cl. A61K 38/24; A61K 9/20

(52) U.S. Cl. CPC A61K 38/24; A61K 9/2072

(57) ABSTRACT

A gonadotropin is administered within a surprisingly effective narrow range for the purpose of treating chronic pain or other central sensitization sequelae. In one aspect, a recipient is provided with at least one of human chorionic gonadotropin (uHCG and/or rHCG), a pharmaceutically active HCG analogue, and a pharmaceutically active metabolite of the HCG or analogue at a dosage selected to provide an amount therapeutically bioequivalent to, a human subcutaneous dosage of between 120 IU/day and 170 IU/day of HCG, and more preferably between 140 IU/day and 160 IU/day of HCG. A combination product is also described, which includes a supply of the HCG-related drug, a delivery device, and a conversion scale for therapeutic bioequivalence that identifies the specific amount and route of administration for chronic pain or central sensitization as an indication of the drug.

This application is a continuation-in-part of U.S. application Ser. No. 14/218,448, filed on Mar. 18, 2014, which is a continuation of U.S. application Ser. No. 13/633,739, filed on Oct. 2, 2012, which is a continuation of pending U.S. application Ser. No. 13/311,250 filed Dec. 5, 2011, which is a continuation-in-part of U.S. patent application Ser. No. 13/211,101 filed Aug. 16, 2011, which claims priority to U.S. Provisional Application No. 61/475,908, filed Apr. 15, 2011, each of which is incorporated herein by reference in its entirety.

### FIELD OF THE INVENTION

The field of the invention is chronic pain management, and more specifically to administration of specific low doses of human chorionic gonadotropin (HCG).

### BACKGROUND

An ongoing and pervasive problem in the medical community is treating patients with chronic pain syndromes. It is well recognized today that chronic pain is fundamentally different from acute pain, also referred to as nociceptive pain, which pain results from a mechanical, chemical, metabolic or inflammatory insult.

It has been recognized by some that since the mechanisms and pathways for chronic and acute pain are physiologically different, they require different approaches for treatment. Unfortunately, many in the medical community continue to treat patients suffering from chronic pain syndromes with agents designed to address acute nociceptive pain pathways. Such methods are often fraught with toxicity and dependence issues, and in the end are generally unsatisfactory in ending pain and/or improving quality of life. A new diagnostic paradigm and treatment protocol is therefore needed to address chronic pain at its root cause.

Central sensitization is a newly recognized diagnostic target entity that underlies a broad range of phenotypic syndromes, including various chronic musculo-skeletal pain, neuropathic pain, and mood and post-traumatic disorders. As used herein, central sensitization means an abnormal state of functioning of the neurons and circuitry of the central pain intensity, perception and modulation systems; due to synaptic, chemical, functional and/or structural changes, in which pain is no longer coupled, as acute nociceptive pain is, to particular peripheral stimuli. Instead, the central nervous system (CNS) initiates, maintains and contributes to the generation of pain hypersensitivity and perception, absent a peripheral stimulus, and ultimately manifests in clinical presentations of phenotypic central sensitivity syndromes (CSS). As used herein, therefore chronic pain and central sensitization represent an overlapping constellation of diagnostic conditions and syndromes.

The present inventors consider the following to be a non-exhaustive listing of conditions associated with (causative or resulting from) central sensitization, each of which is thought to be applicable to humans or other vertebrates.
1. Arachnoiditis
2. Autonomic neuropathies
3. Chronic back pain
4. Chronic joint pain associated metabolic neuropathy
5. Chronic joint pain associated with inflammation
6. Complex Regional Pain Syndrome
7. Fibromyalgia
8. Irritable bowel syndrome
9. Migraine
10. Neuropathic pain
11. Osteoarthritis
12. Post Herpetic neuralgia
13. Post surgical pain syndromes
14. Post Traumatic Stress Disorder Pain Syndrome
15. Rheumatoid, arthritic, psoriatic and other chronic arthropathies
16. Spinal nerve compression syndromes associated with neoplasia and/or disc herniation
17. Trigeminal neuralgia
18. Vulvodynia syndrome

Central sensitization is currently thought to be established via a well characterized constellation of cellular changes termed, neuroplasticity. Neuroplasticity consists of the physical remodeling of neuronal and microglial cyto-architecture; such as changes in synaptic gap junctions, membrane excitability shifts due to ion channel modulation, and gene transcription.

Neuroplasticity changes can be bi-directional. In other words, appropriately functioning cell can undergo remodeling that results in a dysfunctional operating state creating the ‘disease states’ of chronic pain and mood disorders. Conversely, these neuroplasticity mediated dysfunctional changes can be reversed with a return to ‘normal’ functioning, which can correspond clinically to a resolution of a ‘disease’ state.

Central sensitization involves, in part, shifts in gene transcription involved in nociception and pain modulation. Pain, in general, represents a hyper-excitatory state of neuronal tissue associated with an increase in action potential firing. Action potential generation is the result of increased amplitude and/or frequency of electrical signaling. This is created by the cellular integration of changes in molecular signaling, ion gradients and gene expression, ultimately resulting in the perception of acute or chronic discomfort.

Pain transmission and modulation through the central nervous system network of neurons and support glial cells (microglia and astrocytes) is largely under the control of a large family of cellular receptors known as G protein-coupled receptors (GPCRs). The function of these complex trans-membrane receptors is to transduce extracellular stimuli into intracellular signaling including gene transcription. GPCRs modulate and/or mediate virtually all physiologic processes in eukaryotic organisms, including acute and chronic pain. An estimated 90% of all known GPCRs are expressed in the central nervous system. 80% of the currently proposed GPCR families have a known role in modulation of pain. Similarly, most of the identified genes associated with pain modulation are GPCR related genes. Stone L S, Molliver D C. In search of analgesia: Emerging role of GPCRs in pain. Molecular Interventions. 2009 (9):5; 234-251. The LH/HCG receptor is a GPCR. Id.

This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

The LH/HCG receptor complex specifically has been specifically shown to complex with the Gαi/o group resulting in modulation of neurotransmission. Hu L, Wada k, Mores N, Krsmanovic L Z, Catt K J. Essential role of G protein-gated inwardly rectifying potassium channels in gonadotropin-induced regulation of GnRH neuronal firing and pulsatile neurosecretion. Jour Biol Chem. 2006:281(35); 25231-25240. Gαi/o proteins mediate the widespread inhibitory effects of many neurotransmitters and they mediate the effects of almost all analgesic GCPR agonists. Stone L S, Molliver D C. In search of analgesia: Emerging role of GPCRs in pain. Molecular Interventions. 2009 (9):5; 234-251.

Huber, et al has clearly shown this phenomenon occurring at specific HCG concentration levels in endometriotic tissue (1). Some of the specific genes identified in this study were genes encoding for G-protein coupled receptor (GPCR) function (2). See:
1. Huber A, Hudelist G, Knofler N, Saleh L, Huber J C, Singer C F. Effect of highly purified human chorionic gonadotropin preparations on the gene expression signature of stromal cells derived from endometriotic lesions: potential mechanisms for the therapeutic effect of human chorionic gonadotropin in vivo. October 2007 Fertility and Sterility Vol. 88, No. Suppl 2.
2. Foukes T, Wood J N. Pain Genes. PLoS Genetics. July 2008 (4)7:e1000086.

Due to the multiplicity of pathways involved in establishing central sensitization, chronic pain is a complex phenomenon that can be difficult to treat with single-pathway-active-agent therapy. See Latremoliere A, Woolf C J. Central sensitization: A generator of pain hypersensitivity by central neural plasticity. J Pain. 2009 September; 10(9):895-926.

This may explain why there remains a critical dearth of effective medical interventions to treat chronic pain disorders. Traditional pharmaceutical approaches generally deal with a single involved pathway, which tends to yield less than ideal results and is often associated with significant toxicity. For example, the treatment options most commonly investigated to date consist of centrally acting drugs. These include ketamine, dextromethorphan, gabapentin, pregabalin, duloxetine, milnacipran, lamotrigene; and not all of these have reached human trials at this time. Each has demonstrated a poor therapeutic index in trials.

Thus, there is still a need for apparatus, systems, and methods for treating chronic pain, and more generally central sensitization that approaches this phenomenon in a pleiotropic fashion.

### SUMMARY OF THE INVENTION

The inventive subject matter provides apparatus, systems, and methods in which a gonadotropin is administered within a surprisingly effective narrow range for the purpose of treating chronic pain or other central sensitization sequelae in a pleiotropic manner.

In one aspect, contemplated methods involve communicating with a subject, person, non-human animal, or other recipient to determine whether that recipient suffers from chronic pain, and then facilitating the recipient's taking of at least one of human chorionic gonadotropin (HCG), a pharmaceutically active HCG analogue, and a pharmaceutically active metabolite of the HCG or analogue. Preferably, the dosage is selected to provide, or be therapeutically bioequivalent to, a human subcutaneous dosage of between 120 IU/day and 170 IU/day of HCG for the treatment of chronic pain and central sensitization sequalae. More preferably, the dosage is selected to provide, or be therapeutically bioequivalent to, a human subcutaneous dosage of between 140 IU/day and 160 IU/day of HCG.

Contemplated manners of communication include procuring a written and/or oral symptom history, performing physical examination, referring for laboratory tests and other studies, and especially focusing on whether the recipient has one or more of fibromyalgia, irritable bowel syndrome, chronic back pain, chronic arthropathy, inflammatory pain, post herpetic neuralgia, trigeminal neuralgia, neuropathic pain, vulvodynia and migraine. Such communication can be performed synchronously between a health care professional and the recipient, as for example in a doctor's office or over the phone, and/or asynchronously, as for example using physical mail, electronic mail, and so forth. It is also contemplated to conduct at least a physical test that aids in distinguishing between nociceptive pain and central sensitization that the recipient may have.

Contemplated manners of facilitating the recipient's taking of the drug(s) include administering the drug(s) as a drug product or combination product, issuing a prescription for the drug(s) in a combination product, suggesting use of the drug(s), as in a book or article, and/or providing the recipient (directly or indirectly) with contact information for a supply of the drug(s) as a drug product. It is contemplated that one or more of the drugs could be self-administered by the recipient as a combination product with therapeutic bioequivalent safety and efficacy.

The drug(s) are preferably used as a mono-therapy for the central sensitization, but could be combined with other drugs and/or non-drug treatments, including for example, life-style changes such as elimination diet, and anti-inflammatory diet. It is preferred that the drug(s) is/are taken in the absence of concurrent opioid pain treatment, and in the absence of concurrent treatment with another gonadotropic substance. However, when the drug(s) product is/are taken with concomitant opioids the recipient is enabled to decrease gradually and/or discontinue the opioid daily requirement, while continuing to benefit from the main analgesic and mood improving effect of the drug(s) product.

In some instances, a clinician or other provider may have been administering or recommending HCG for some other purpose, or in some other dosage, not realizing that HCG can be effective to ameliorate chronic pain or central sensitization sequelae as claimed herein. In such instances it is contemplated that the provider receive information that, HCG as a drug product delivered subcutaneously has a peak therapeutic effect on central sensitization between 120 IU/day and 170 IU/day, inclusive, and can thereafter administer or recommend HCG, a pharmaceutically active HCG analogue, or a pharmaceutically active metabolite of the HCG or analogue as claimed herein, as a drug product or combination product with therapeutic bioequivalent efficacy and safety.

It is contemplated that a combination product could include (a) a supply of a drug selected from the group consisting of at least one of HCG, a pharmaceutically active HCG analogue, and a pharmaceutically active metabolite of the HCG or analogue, (b) a delivery device and (c) a therapeutic scale that identifies (or selects/matches) the type of HCG, the route of administration and (with) the amount of drug to-be-delivered in order to safely and effectively treat at least one of chronic pain and central sensitization as an indication for the drug.

In some aspects of preferred embodiments, the therapeutic scale identifies a daily dosage regimen of the drug to-be-delivered through the selected route of administration in reference to a subcutaneous dosage of human chorionic gonadotropin (HCG) between 120 IU/day and 170 IU/day, inclusive, with respect to chronic pain relief and other central sensitization sequelae relief In other aspects of some preferred embodiments, the therapeutic scale identifies a daily dosage regimen of the drug to-be-delivered through the selected route of administration in reference to a subcutaneous dosage of human chorionic gonadotropin (HCG) between 140 IU/day and 160 IU/day, inclusive, with respect to chronic pain relief and other central sensitization sequelae relief

In other aspects of preferred embodiments, a combination product can include a vial or cartridge with at least first chamber having a lyophilized preparation of the drug that is suitable for injection when mixed with a diluent contained in a second chamber of the cartridge. Alternatively, the drug could be provided as part of a combination product in a stabilized liquid form. The drug could be disposed in an auto-inject or a dial up dosing pen equipped with a cartridge with at least first and second chamber for HCG formulation storage and delivery. Alternatively or additionally, a combination product could include a container that houses the drug in an orally available composition such as oral disintegrating tablets dispenser or as an aerosolized nasal spray. The combination product could also consist of a sub-dermal pellet, a device for placement of said pellet for a timed release of HCG formulation, and a therapeutic scale.

A provider need not actively communicate with the recipient, but could determine in some other manner that the recipient might suffer from a central sensitization disorder. For example, the step of determining could comprise initiating a plurality of testing procedures that includes: (a) at least one test selected from a first group consisting of dynamic tactile allodynia, secondary punctate/pressure hyperalgesia, temporal summation, and sensory after effects, and (b) at least one other test selected from a second group consisting of SMAC 25, fMRI, Neuro-Endocrine profile (neurotransmitters and hormones), CSF study (substance P, glutamate, NGF, BDNF), cytokines profile, genetic polymorphism profile, food allergy panel, and heavy metals analysis panel.

In other aspects, the step of determining comprises (i) determining that the subject may suffer from central sensitization due to a trauma, and (ii) providing the subject with access to the drug peritraumatically. In yet other aspects of some preferred embodiments, the trauma is a surgery, and the drug is administered perioperatively.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of one preferred embodiment of a method of interacting with a person.

FIG. 2 is a perspective view of one preferred embodiment of a combination product that includes a supply of a drug, a delivery device and a therapeutic scale.

FIG. 3 is a perspective view of delivery device as an auto-inject dosing pen.

FIG. 4 is a perspective view of a delivery device as a dial-up dosing pen.

FIG. 5 is a schematic of one preferred embodiment of a method of treating a subject.

### DETAILED DESCRIPTION

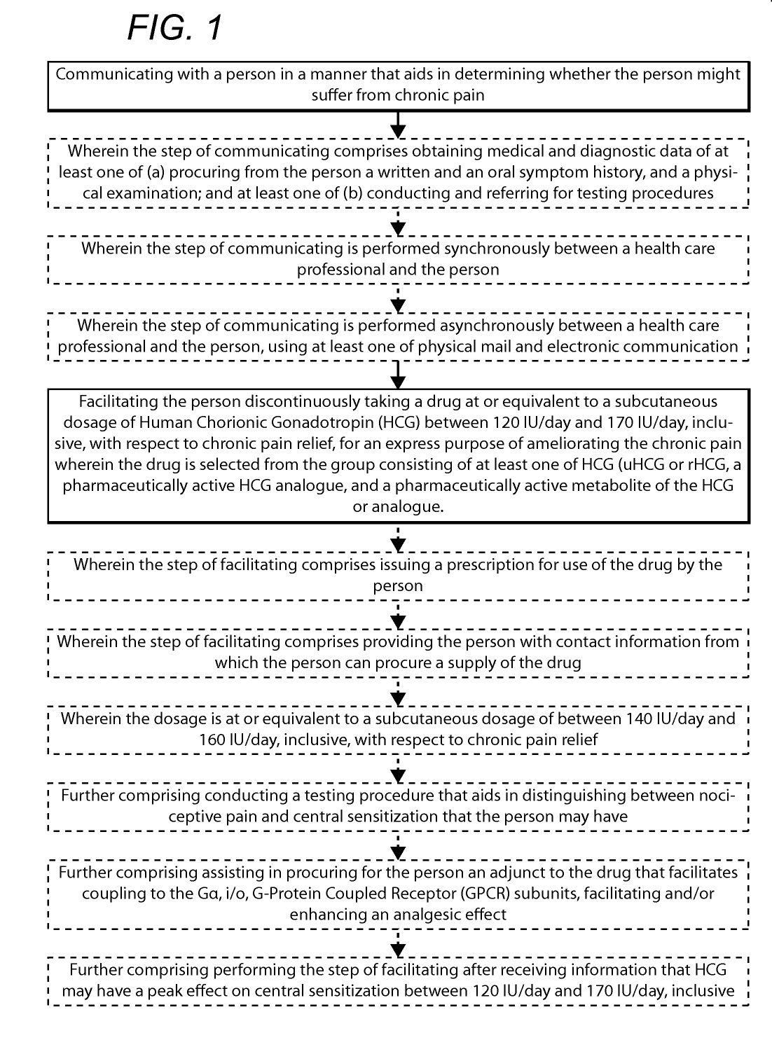

In FIG. 1 a schematic of a method of interacting with a person is shown. The method includes the steps of: (i) communicating with the person in a manner that aids in determining whether the person might suffer from chronic pain; and (ii) facilitating the person discontinuously taking a drug according to the therapeutic scale of a subcutaneous dosage of human chorionic gonadotropin (HCG) between 120 IU/day and 170 IU/day, inclusive, with respect to chronic pain relief and central sensitization disorders, for an express purpose of ameliorating the chronic pain or central sensitization, wherein the drug is selected from the group consisting of at least one of HCG, a pharmaceutically active HCG analogue, and a pharmaceutically active metabolite of the HCG or analogue. Alternatively, a therapeutic dosage for oral mucosa administration can be used in amount of 500 IU/day to 2000 IU/day, inclusive, for an express purpose of ameliorating the chronic pain or central sensitization.

As used herein, the term human chorionic gonadotropin (HCG) means a compound in a pharmaceutical composition of matter obtained from at least one of the following sources: purified urine of pregnant and/or post-menopausal women (uHCG); purified bacterial, yeast, plant and/or mammalian cell cultures utilizing recombinant DNA hybridization techniques (rHCG).

As used herein, the term pharmaceutically active HCG analogue means a compound that, with respect to amelioration of chronic pain or other sequelae of central sensitization, has either (i) at least a partial biological activity of HCG (e.g., mutant, truncated form, chemically modified), or (ii) can bind to HCG receptors, either an agonist or neutral ligand. As used herein the term “analogues” includes prodrugs of HCG.

As used herein, a “prodrug” means a modification of a contemplated compound, wherein the modified compound exhibits less pharmacological activity (as compared to the contemplated compound) and wherein the modified compound is converted within a target cell (e.g., hepatic-cell) or target organ/anatomic structure (e.g., spinal cord) back into the contemplated form. For example, conversion of contemplated compounds into prodrugs may be useful where the active drug is too toxic for safe systemic administration, or where the contemplated compound is poorly absorbed by the digestive tract or oral mucosa or other compartment or cell, or where the body breaks down the contemplated compound before reaching its target. Thus, it should be recognized that the compounds according to the inventive subject matter can be modified in numerous manners, and especially preferred modifications include those that improve one or more pharmacokinetic and/or pharmacodynamic parameter. For example, one or more substituents may be added or replaced to achieve a higher area under the curve (AUC) of HCG in serum.

As used herein, the term pharmaceutically active metabolite means any compound resulting from in vivo metabolism of HCG or an HCG analogue (for example, via proteolytic digest, glycosylation, sialation, hydroxylation, phosphorylation, sulfuration, etc), where the metabolite is therapeutically effective and safe with respect to amelioration of chronic pain or other sequelae of central sensitization.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The step of communicating can comprise obtaining medical and diagnostic data, including procuring from the person at least one of a written and an oral symptom history, performing physical examination; and referring for tests and imaging studies. The symptom history can be used to assist in determining whether the person has at least one of central sensitization disorders, fibromyalgia, irritable bowel syndrome, chronic arthropathy, inflammatory pain, neuropathic pain, chronic back pain, post herpetic neuralgia, post-surgical pain syndrome, arachnoiditis, trigeminal neuralgia, vulvodynia and migraine. Specifically, it is preferred that the subject's symptom history is procured with clinical tools recognized to be useful in determining the presence of central sensitization syndromes (CSS) such as central sensitization inventory (CSI) and /or quantitative sensory testing (QST). The CSI is a validated self-report screening instrument that can assist in identifying a subject with CSS. Mayer T G, Neblett R, Cohen H, Howard K J, Choi Y H, Williams M J, Perez Y, Gatchel R J. The Development and Psychometric Validation of the Central Sensitization Inventory (CSI)_Pain Pract. 2012 April; 12(4): 276-285.

The step of communicating can be performed either synchronously between a health care professional and the person, or alternatively, asynchronously between a health care professional and the person using physical mail or electronic communication.

The step of facilitating can comprise issuing a prescription for use of the combination product by the person. In addition, the step of facilitating can comprise providing the person with contact information from which the person can procure a supply of the drug as a combination product.

The dosage for each route of administration is preferably selected with the therapeutic scale in reference to a subcutaneous dosage of between 140 IU/day and 160 IU/day, inclusive, with respect to chronic pain relief

The method of FIG. 1 can further include the step of conducting and/or referring for a physical testing procedures that aids in distinguishing between nociceptive pain and central sensitization that the person may have. For example, quantitative sensory testing can serve this function as described in U.S. Pat. No. 8,652,189 B2 and Pub. No.: US 2011/0082384 A1

The method can also include the step of assisting in procuring the drug for the person as a mono-therapy for the central sensitization. In addition, the method can include the step of assisting in procuring a composition for the person in adjunct to the drug that facilitates coupling to the Gαi/o, G-Protein Coupled Receptor (GPCR) subunits, facilitating and/or enhancing an analgesic effect.

It is also contemplated that one can perform the step of “facilitating” after receiving information that HCG, delivered subcutaneously, has a peak effect on central sensitization between 120 IU/day and 170 IU/day, inclusive.

A therapeutic scale of a subcutaneous dosage of HCG can include all suitable modes of administration, such as intramuscularly, sub-dermal, oral dissolving tablet, sublingual as a liquid, transdermal, rectal, and via sub-dermal slow release pellets. U.S. Pat. No. 6,488,649 teaches suitable sub-dermal pellet implant devices.

For example, for the oral dissolving tablet drug product, the therapeutic scale identifies an oral daily dosage unit of HCG in a range from about 500 IU/day to about 2000 IU/day for optimal analgesic effect and minimal toxicity signs. In some embodiments, the oral daily dosage unit of HCG is in a range from about 1000 IU/day to about 2000 IU/day, from about 1100 IU/day to about 1900 IU/day, from about 1200 IU/day to about 1800 IU/day, from about 1300 IU/day to about 1700 IU/day, from about 1400 IU/day to about 1600 IU/day, from about 500 IU/day to about 1500 IU/day, from about 500 IU/day to about 1400 IU/day, from about 500 IU/day to about 1300 IU/day, from about 500 IU/day to about 1200 IU/day, from about 500 IU/day to about 1100 IU/day, from about 600 IU/day to about 1500 IU/day, from about 700 IU/day to about 1500 IU/day, from about 750 IU/day to about 1500 IU/day, from about 800 IU/day to about 1500 IU/day, from about 900 IU/day to about 1500 IU/day, from about 1000 IU/day to about 1500 IU/day, from about 600 IU/day to about 1400 IU/day, from about 600 IU/day to about 1300 IU/day, from about 600 IU/day to about 1200 IU/day, from about 600 IU/day to about 1100 IU/day, from about 700 IU/day to about 1700 IU/day, from about 700 IU/day to about 1600 IU/day, from about 700 IU/day to about 1400 IU/day, from about 700 IU/day to about 1300 IU/day, from about 700 IU/day to about 1200 IU/day, from about 700 IU/day to about 1100 IU/day, from about 800 IU/day to about 1400 IU/day, from about 800 IU/day to about 1300 IU/day, from about 800 IU/day to about 1200 IU/day, from about 800 IU/day to about 1100 IU/day, from about 800 IU/day to about 1000 IU/day, from about 900 IU/day to about 1100 IU/day, from about 1000 IU/day to about 1200 IU/day, and/or from about 950 IU/day to about 1050 IU/day. For example, the oral daily dosage unit of HCG can be about 1000 IU/day. In some embodiments, the oral daily dosage unit of HCG is about 1500 IU/day.

The method shown in FIG. 1 can be used to treat numerous disorders related to chronic pain and central sensitization. For example: fibromyalgia, rheumatoid arthritis, osteoarthritis, chronic arthropathy, spinal nerve compression syndromes associated with neoplasia and/or disc herniation, chronic back pain, chronic joint pain of any etiology associated with inflammation and/or structural joint abnormalities, post herpetic neuralgia, trigeminal neuralgia, chronic metabolic neuropathy associated with chronic pain, migraine, inflammatory pain, post surgical pain syndromes including phantom limb pain, Post Traumatic Stress Disorder, Irritable Bowel Syndrome, autonomic neuropathies, arachnoiditis, Chronic Regional Pain Syndrome, Vulvodynia, and chronic pain syndrome associated with activation of central sensitization pathways.

It is also contemplated that the “person” can include humans, pets, and mammals.

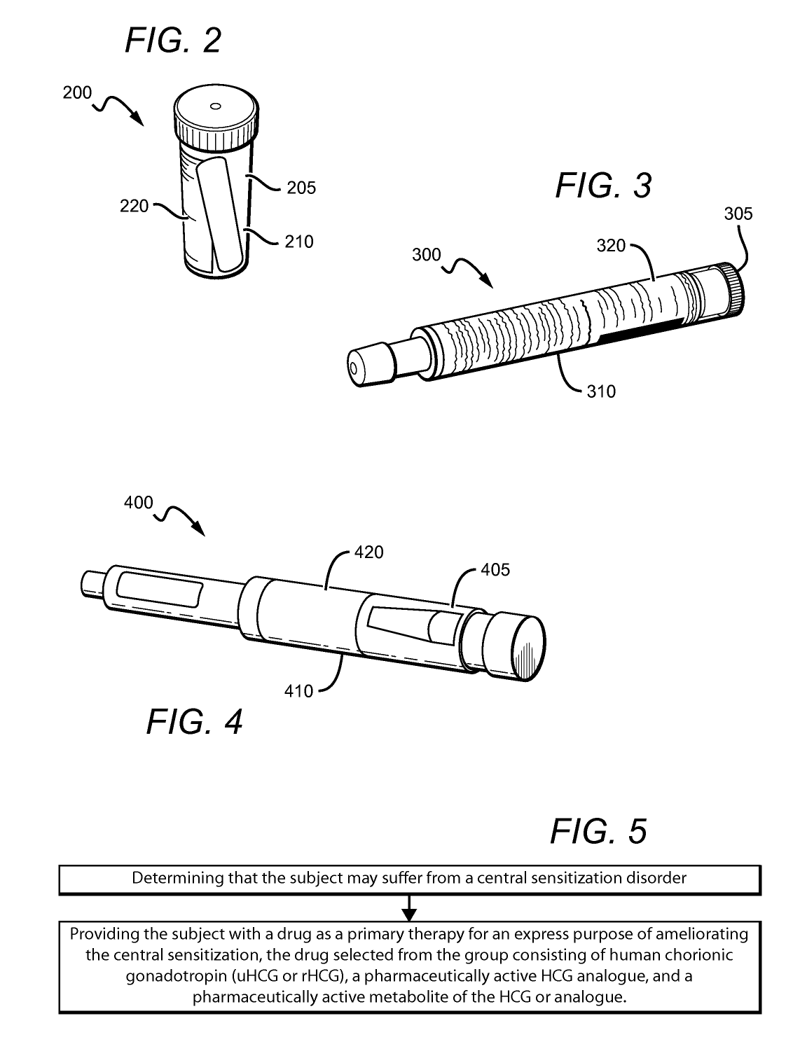

FIG. 2 one preferred embodiment of a combination product 200. Combination product 200 includes a dispenser 205. Dispenser 205 is a delivery device that holds a drug 210 and an exterior/guiding therapeutic scale of chronic pain and central sensitization relief 220. Drug 210 is preferably selected from the group consisting of at least one of HCG (uHCG and/or rHCG), a pharmaceutically active HCG analogue, and a pharmaceutically active metabolite of the HCG or analogue. Drug 210 is part of a sublingual or an oral dissolving tablet drug product. It is also contemplated that drug 210 could comprise other drug product formulations.

### Administration

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means in conjunction with the methods described herein, and, if desired, the human chorionic gonadotropin may be mixed with any conventional additive or excipient, such as a binder, a disintegrating agent, a lubricant, a corrigent, a solubilizing agent, a suspension aid, an emulsifying agent, or a coating agent in addition to a cyclodextrin and a buffer. Although the dosage will vary depending on the symptoms, age and body weight of the patient, the nature and severity of the disorder to be treated or prevented, the route of administration and the form of the drug, in general, a daily dosage of from 0.01 to 5000 IU of the human chorionic gonadotropin is recommended for an adult human patient, and this may be administered in a single dose or in divided doses. The amount of active ingredient which can be combined with a carrier material to produce a single dosage form will generally be that amount of the compound which produces a therapeutic effect. In general, compositions intended for parenteral use (e.g., intravenous, subcutaneous injection) include a substituted cyclodextrin. Compositions administered via other routes, particularly the oral route, include a substituted or unsubstituted cyclodextrin.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions provided herein are non-pyrogenic, i.e., do not induce significant temperature elevations when administered to a patient.

The term “pharmaceutically acceptable salt” refers to the relatively non-toxic, inorganic and organic acid addition salts of the human chorionic gonadotropin. These salts can be prepared in situ during the final isolation and purification of the human chorionic gonadotropin, or by separately reacting human chorionic gonadotropin in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, the human chorionic gonadotropin provided herein may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of human chorionic gonadotropin as an active ingredient. A composition may also be administered as a bolus, electuary, or paste.

In solid dosage forms for oral administration (capsules, tablets, pills, dragees, powders, granules, and the like), the human chorionic gonadotropin is mixed with one or more pharmaceutically acceptable carriers, such as sodium citrate or dicalcium phosphate, and/or any of the following: (1) fillers or extenders, such as starches, cyclodextrins, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Examples of oral formulations include those described in U.S. Pat. Nos. 7,122,198 and 7,605,122, each of which is hereby incorporated by reference in its entirety.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active human chorionic gonadotropin may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

It should be recognized that all formulations are deemed suitable for use herein and especially include parenteral and oral formulations. For example, for oral administration, contemplated compositions may be in the form of, e.g., a tablet (e.g., an orally dissolving tablet), capsule, suspension, or liquid. For an orally dissolving tablet, the pharmaceutical composition is preferably made in the form of a dosage unit containing a particular amount of the active ingredient, such as 250 IU, 500 IU or 1000 IU of HCG, and excipients as accelerators for disintegration of the tablet, such as glycolate forms (sodium starch) cellulose forms (microcrystalline, sodium or calcium carboxy-methyl); and other excipients such as flavors (peppermint, vanilla), fillers (mannitol, sorbitol, xylitol, pregelatinized starch), surface active agents (sodium dodecyl sulfate, sorbital fatty acid esters), binder (PVP, PVA), lubricants (stearic acid and stearates, talc, colloidal silicon dioxide). For parenteral formulation, the active ingredient can be administered by injection as a lyophilized or a stabilized liquid composition wherein, for example, saline, sucrose, maltose or water may be used as a suitable carrier. In some embodiments, it is contemplated that the formulation is suitable for intrathecal administration, subdermal pellets, administration via aerosol, and for topical administration. Consequently, where the compound is formulated for intrathecal administration (e.g., in the treatment of spinal cord injury), it is preferred that the compound is prepared as an injectable solution, suspension, or emulsion. Alternatively, in contemplated formulations, contemplated compounds may be formulated for aerosol delivery (e.g., micropowderized, coated onto a dispersible carrier, dissolved in atomizable solvent, etc.) and slow-release pellets for subdermal implant. Furthermore, especially suitable formulations may be sterile aqueous solutions for topical spray or drop administration, or application as a tincture. In some embodiments, suitable topical formulations may include creams, ointments, foams, lotions, emulsions, etc.

It should be appreciated that the choice of the particular formulation and carrier will at least in part depend on the specific use and type of compound. There are numerous manners of drug formulation known in the art, and all of those are deemed suitable for use herein (see e.g., Pharmaceutical Preformulation and Formulation: A Practical Guide from Candidate Drug Selection to Commercial Dosage Form by Mark Gibson; Informa HealthCare, ISBN: 1574911201; or Advanced Drug Formulation Design to Optimize Therapeutic Outcomes by Robert O. Williams, David R. Taft, and Jason T. McConville; Informa HealthCare; ISBN: 1420043870).

Although age, gender and weight of a recipient of HCG treatment for chronic pain does not appear with current studies to affect the preferred therapeutic ranges, it is contemplated that the amount of therapeutically active compound that is administered and the dosage regimen for treating a disease condition with the compounds and/or compositions of this invention could depend on one of more of a variety of factors, including the age, weight, sex and medical condition of the subject, the severity of the disease, the route and frequency of administration, and the particular compound employed, and thus may vary widely. Hence, the need for a combination product with a conversion scale for therapeutic bioequivalence in reference to the safe and efficacious subcutaneous dosage range in relieving chronic pain and central sensitization disorders.

The therapeutic scale 220 identifies at least one route of administration of the drug for chronic pain and central sensitization as an indication for the drug. Therapeutic scale 220 also identifies a daily dosage unit and regimen in reference to a subcutaneous dosage unit of human chorionic gonadotropin (HCG) between 120 IU/day and 170 IU/day, inclusive, with respect to relief of chronic pain or central sensitization sequelae. Alternatively, conversion scale for therapeutic bioequivalence 220 could identify a daily dosage regimen in reference to a subcutaneous dosage of human chorionic gonadotropin (HCG) between 140 IU/day and 160 IU/day, inclusive, with respect to relief of chronic pain or central sensitization sequelae.

Combination product 200 could also include a vial or cartridge with a first chamber, which contains a lyophilized preparation of the drug that is suitable for injection when mixed with a diluent contained in a second chamber of the cartridge. Alternatively, the combination product could contain a stabilized liquid form of the drug. The drug could be stabilized in any suitable manner, as for example, using methods set forth in US006706681B1.

FIG. 3 shows a combination product 300, which includes an auto-inject dosing pen 305. Pen 305 is a delivery device that has a drug 310 and a therapeutic scale 320. Drug 310 is similar to drug 210 except that drug 310 is in a stabilized liquid or lyophilized form. Therapeutic scale 320 is similar to therapeutic scale 220, except that it refers to subcutaneous rather than oral administration.

FIG. 4 shows a combination product 400, which includes a dial-up dosing pen 405. Pen 405 is a delivery device that has a stabilized liquid or lyophilized form of drug 410 and an exterior therapeutic scale 420. Drug 410 is similar to drug 310. Therapeutic scale 420 is similar to therapeutic scale 220, except that it refers to subcutaneous rather than oral route of administration.

In FIG. 5, a schematic of a method of treating a subject is shown. The method includes the steps of: (i) determining that the subject may suffer from a central sensitization disorder; and (ii) providing the subject with a combination product as a primary therapy for an express purpose of ameliorating the central sensitization. The drug is either human chorionic gonadotropin (uHCG or rHCG), a pharmaceutically active HCG analogue, or a pharmaceutically active metabolite of the HCG or analogue.

The step of determining that the subject may suffer from central sensitization disorder can include initiating a plurality of testing procedures. Such laboratory tests may include: (i) at least one test selected from a first group consisting of dynamic tactile allodynia, secondary punctate/pressure hyperalgesia, temporal summation, and sensory after effects, and (b) at least one other test selected from a second group consisting of SMAC 25, fMRI, Neuro-Endocrine profile (neurotransmitters and hormones), CSF study (substance P, glutamate, NGF, BDNF), cytokines profile, genetic polymorphism profile, food allergy panel, and heavy metals analysis panel.

For example, for the first group one could select a quantitative sensory testing as described in U.S. Pat. No. 8,652,189 and US application 2011/0082384.

In addition, the step of determining that the subject may suffer from central sensitization disorder can include: (i) determining that the subject may suffer from central sensitization due to a trauma, and (ii) providing the subject with access to the drug peritraumatically. The trauma can include a surgery, traumatic brain injuries (TBI), stroke, transient ischemic attack (TIA), motor vehicle accident, gunshot would, industrial accidents, assault, blunt trauma, repetitive traumatic sports injuries, eventful psycho-emotional trauma (rape, war, natural catastrophes, but not limited to this list).

It is especially contemplated that peri-traumatic administration can include perioperative administration. For example, for surgical procedures known to be associated with significant long term pain syndromes (i.e., thoracotomy, mastectomy and amputations) post-operatively; where the drug is administered peri-operatively in an effort to prevent the development of central sensitization mediated post-op chronic pain syndromes; or alternatively, the drug can be administered to those with established central sensitization mediated post-operative pain for analgesia.

The step of providing the subject with a combination product can include suggesting and/or instructing the subject to self-administer an amount of the drug therapeutically bioequivalent to a subcutaneous dosage of human chorionic gonadotropin (HCG) between 120 IU/day and 170 IU/day, inclusive, with respect to chronic pain relief.

The method in FIG. 5 preferably does not include concomitantly treating the subject with an opioid pain treatment or another gonadotropic substance.

### Case Study

### Materials/Methods

Based on previous practice experience with pain control and established safety with human use of HCG (see The role of hCG in reproductive medicine. BJOG: an International Journal of Obstetrics and Gynaecology. November 2004, Vol. 111, pp. 1218-1228), the current inventors aimed at determining the efficacy of clinical use in a standardized fashion in a representative series of 24 patients (patient characteristics are presented in Table 1).

TABLE 1
Demographics, Diagnosis, and Treatment Group
According to Each Patient of Study

| Patient | Age | | | |
|---|---|---|---|---|
| # | (years) | Gender | Diagnosis | Treatment Group |
| 1 | 46 | Female | Disc pain | HCG Weight Loss |
| 2 | 56 | Female | Fibromyalgia | HCG Weight Loss |
| 3 | 56 | Female | Disc pain | HCG Weight Loss |
| 4 | 41 | Female | Headache | HCG Weight Loss |
| 5 | 45 | Female | Back pain | HCG Weight Loss |
| 6 | 41 | Female | Disc pain | HCG Weight Loss |
| 7 | 52 | Female | Fibromyalgia | HCG Weight Loss |
| 8 | 53 | Female | Osteoarthritis | HCG Weight Loss |
| 9 | 60 | Female | Osteoarthritis | HCG Weight Loss |
| 10 | 66 | Female | Fibromyalgia | HCG Weight Loss |
| 11 | 61 | Female | Neuralgia | HCG Weight Loss |
| 12 | 78 | Female | Osteoarthritis | HCG Weight Loss |
| 13 | 61 | Female | Fibromyalgia/ | HCG Weight Loss |
| | | | Rheumatoid | |
| | | | Arthritis | |
| 14 | 30 | Male | Osteoarthritis | HCG Weight Loss |
| 15 | 53 | Male | Disc pain | HCG Pain |
| 16 | 53 | Female | Osteoarthritis | HCG Pain |
| 17 | 59 | Male | Osteoarthritis | HCG Pain |
| 18 | 42 | Female | Osteoarthritis | HCG Pain |
| 19 | 47 | Female | Rheumatoid | HCG Pain |
| | | | Arthritis | |
| 20 | 47 | Female | Disc pain | HCG Pain |
| 21 | 44 | Female | Fibromyalgia | HCG Pain |
| 22 | 19 | Female | Osteoarthritis | HCG Pain |
| 23 | 43 | Male | Osteoarthritis | HCG Pain |
| 24 | 76 | Female | Osteoarthritis | HCG Pain |

The majority of patients listed were enrolled in an HCG weight loss program that required a 500 cal/day diet along with daily injections of 150 IU/day of HCG subcutaneously for a six week period. HCG was provided to patients in a lyophilized powdered form in 5,000 IU or 10,000 IU vials for reconstitution prior to use and requiring refrigeration after mixing to maintain potency (HCG 5000 IU vials, KRS Global, Boca Raton, Fla.; HCG 10,000 IU vials, brand name Abraxis™; HCG 10,000 IU vials, brand name Pregnyl™). All HCG was sourced from major suppliers in China coming to the US from FDA approved and CGM P (Current Good Manufacturing Practices) compliant facilities. HCG from these facilities is either sourced from the urine of pregnant women (u-HCG) or from recombinant DNA (r-HCG) production protocols.

Patients were allowed to continue all prescribed medications during the diet, and were all placed on a specific nutritional support regimen which included a daily blend of multi-vitamins, digestive enzymes, probiotics, amino acids, potassium, magnesium, calcium, and 1-carnitine.

All cases referenced except two required re-institution of daily subcutaneous injections of 150 IU HCG for pain control after a relapse of the pain subsequent to completion of the weight loss program and daily HCG administration. Most patients returned to the office several weeks after completion of the weight loss program to report a return of pain and a desire to restart treatment. Universally, reinstitution of HCG treatment resulted in an identical clinical response that persisted in all patients as long as treatment was) continued. None of the patients, to date, has developed tachyphylaxis or need for additional pain control measures.

Patients were carefully monitored for any treatment toxicity including transient hair loss and signs or symptoms of ovarian hyper-stimulation syndrome (OHS). However, accumulated clinical experience (e.g., The role of hCG in reproductive medicine. BJOG: an International Journal of Obstetrics and Gynaecology. November 2004, Vol. 111, pp. 1218-1228) indicate that such expected toxicities are usually associated with higher dosage HCG of above 1500 IU/day. The current inventors have observed no evidence of toxicity in any patients on continued treatment.

Patient responses were quantified utilizing the DoloTest™; a validated health-related quality of life (HRQoL) tool for pain patients that evaluates not only pain but also other areas of quality of life that chronic pain often severely impacts. (See Kristiansen K, Lyhgholm-Kjaerby P, Moe C. Introduction and Validation of DoloTest: A Health Related Quality of Life Tool Used in Pain Patients. Pain Practice 2010 September-October; 10(5):396-403]]

Patients responded to questions posed by the investigators regarding functioning level both before HCG exposure and after institution of HCG treatment. As responses were not graded in any way over time, there were only two end points that were evaluated: (1) on and (2)off HCG treatment.

In all instances there has been a significant or complete elimination of need for any additional pain control interventions. The need for continued visits to physicians' offices for medical, interventional pain management or chiropractic therapy was eliminated. Most importantly, DoloTest™ indicators were all substantially improved for all patients listed. All patients answered all questions.

### Results

SPSS v15.0 was used for all descriptive and inferential analyses. (See Table 2)

A total of 10 analyses were performed with multiple analyses being performed on the same data. To adjust for the increased chance of a Type I error, a Bonferroni correction was done to adjust the significance level for rejection of the Null hypothesis. Using this method, rejection of the Null hypothesis was determined for all analyses when the p-value was less than or equal to 0.005 (See Table 2).

A series of eight 2X2 mixed-ANOVA analyses were performed on the data. The between groups variable for all analyses was the patient group with two categories as mentioned above, and the within group independent variable was time; with two categories of (a) pre-treatment DoloTest score, and (b) post-treatment DoloTest score.

For each of the eight DoloTest™ domains analyzed for all patients in both groups, scores were significantly lowered or improved post-HCG treatment, with p-values<0.0005 for each domain analyzed (See Table 2).

### Pain and Mood:

Finally, two constructs were derived from the DoloTest™ instrument: (a) Physical Aspect and (b) Spiritual, Social and Sleep Aspect. Physical Aspect included: Problems With Light Physical Activities, Problems with Strenuous Physical Activities, Problems Doing Your Job, and Reduced Energy and Strength. Social, Spiritual and Sleep Aspect included: Low Spirit, Reduced Social Life and Problems Sleeping.

For each group, the summed score for each category was divided by the number of variables to obtain an average score for both pre and post treatment with HCG. A Pearson's correlation was then performed on the mean difference scores of the two constructs or groups to assess a linking of these two aspects of chronic pain fallout. Results were statistically significant (p<0.0005) suggesting a strong direct correlation, as we would expect, between the two constructs or groups, i.e., when scores increase or decrease for the physical aspect, scores move in a parallel fashion for the Spiritual/Social/Sleep construct. Clearly, a direct correlation between the pain index and other DoloTest™ items score was seen both in the pre-treatment and the post-treatment groups. As noted above, and of particular interest, is the fact that for most of these patients (91.66%), the need for continued adjuvant treatment for pain, in addition to continued HCG administration, was eliminated.

For all patients listed, the time required to see a positive clinical response was immediate and within a 1-2 day window. For most patients treated, there was a need for continued HCG administration for maintenance of a continued response. In these patients, discontinuing HCG has consistently resulted in a return of their pain syndrome, at what was perceived to be the pre-treatment level. Re-instituting HCG therapy at 150 IU/day subcutaneously resulted promptly in pain relief within 1-2 days.

To date, no patient requiring continuous HCG administration for pain control has reported any decrease in response or need for additional pain treatment. The longest term patient in this series with such consistent response is Patient #9, now at 24 months.

Patients #11 and #13 both manifested a pain relief response in the context of their initial HCG weight loss cycle which continued to be fully sustained without the need for continued HCG administration or further clinical pain interventions. Patient #11 had a severe post herpetic neuralgia requiring multiple daily doses of narcotics that resolved completely with a single six week exposure to HCG and Patient #13 suffered from fibromyalgia and RA requiring weekly Enbrel™ injections and subsequently she has not required treatment for over a year since her initial HCG exposure.

For the remainder of patients, daily dosing of HCG at 140 -170 IU/day has been required for continued pain control.

TABLE 2
MEASURES OF CENTRAL TENDENCY FOR PRE-
TREATMENT AND POST-TREATMENT DOLOTEST ™
ITEM SCORES FOR THE SAMPLE OVERALL
(N = 24)

| DoloTest Item ™ | M | SD | Mdn | Sample Range |
|---|---|---|---|---|
| Pain | | | | |
| Pre-Treatment | 65.83 | 18.91 | 64.50 | 27-97 |
| Post-Treatment | 15.00 | 12.32 | 11.50 | 0-50 |
| Problems with light | | | | |
| physical activities | | | | |
| Pre-Treatment | 60.17 | 24.65 | 65.00 | 12-98 |
| Post-Treatment | 9.38 | 14.47 | 1.00 | 0-58 |
| Problems with more strenuous | | | | |
| physical activities | | | | |
| Pre-Treatment | 75.29 | 17.50 | 74.50 | 41-98 |
| Post-Treatment | 20.21 | 15.93 | 15.50 | 0-50 |
| Problems doing your job | | | | |
| Pre-Treatment | 36.21 | 28.70 | 35.5 | 0-95 |
| Post-Treatment | 4.50 | 8.30 | 0.00 | 0-29 |
| Reduced energy and strength | | | | |
| Pre-Treatment | 69.29 | 15.08 | 66.5 | 37-98 |
| Post-Treatment | 13.25 | 15.25 | 8.00 | 0-50 |
| Low spirit | | | | |
| Pre-Treatment | 67.21 | 21.09 | 70.50 | 6-98 |
| Post-Treatment | 12.00 | 12.41 | 9.00 | 0-51 |
| Reduced social life | | | | |
| Pre-Treatment | 58.42 | 27.02 | 67.50 | 10-98 |
| Post-Treatment | 11.00 | 12.72 | 7.50 | 0-50 |
| Problems sleeping | | | | |
| Pre-Treatment | 66.13 | 23.75 | 73.00 | 18-97 |
| Post-Treatment | 11.04 | 11.46 | 9.50 | 0-50 |

Note.
M = Mean; SD = Standard Deviation; Mdn = Median.
Possible Range for each DoloTest ™ item is 0-100.

### Discussion

One of the most interesting aspects of the work of the current inventors is that HCG appears to be very effective in treating chronic pain and other sequelae of central sensitization at 120 IU/day -170 IU/day subcutaneously, and more especially 140 IU/day -160 IU/day subcutaneously, but not at other dosages. Thus, at subcutaneous dosages of 200 IU/day, 300 IU/day and 500 IU/day are all markedly less safe and effective, and lower dosages are also markedly less effective. This seems to be empirically true regardless of age (at least for adults), gender, weight, and other factors.

In a small number of patients diagnosed with chronic pain associated with central sensitization, including neuropathic pain, arachnoiditis, fibromyalgia, chronic back pain, osteoarthritis, post-surgical-pain syndrome has been found that administration of an oral dissolving tablet formulation of HCG approached therapeutic effectiveness of the subcutaneous administration of HCG. A therapeutic response has been recorded in five patients when administered daily dose of HCG with an oral dissolving tablet formulation between 500 IU/day and 2000 IU/day. Three of these patients have reported pain relief and other health benefits at 1500 IU/day as being comparable with the subcutaneous injection of 150 IU/day. These individual cases suggest that a preferred range of 1000 IU/day to 1500 IU/day for oral dissolving tablet formulation of HCG for treatment of chronic pain associated with central sensitization.

Although this specification and the appended claims should not be limited by the validity of any particular theory or mechanism of action, the current inventors have contemplated a theoretical framework that may have significant validity in explaining the surprising results discussed herein.

HCG is now recognized to have pleiotropic actions throughout the body as evidenced by the documented presence of receptors for HCG in multiple cellular compartments including the CNS. See Rao C V. An overview of the past, present, and future of non-gonadal LH/hCG actions in reproductive biology and medicine. Semin Reprod Med, 2001; 19:7-17; and Lei Z M, Rao C V. Neural actions of luteinizing hormone and human chorionic gonadotropin. Semin Reprod Med, 2001; 9:103-109. The exact functioning of these receptors is not fully elucidated but indications regarding their putative functions have been delineated as the current inventors will cite below.

In the adult CNS, HCG receptors have been established to be present in the hippocampal formation, hypothalamus, cerebral cortex, brain stem, cerebellum, pituitary gland, neural retina, spinal cord and the ependymal region (Lei Z M, Rao C V, Kornyei J L, Licht P, Hiatt E S. Novel expression of human chorionic gonadotropin/luteinizing hormone receptor gene in brain. Endocrinology, 1993; 132:2262-2270). Both neurons and glial cells are shown to express receptors for HCG (Lei Z M, Rao C V, Kornyei J L, Licht P, Hiatt E S. Novel expression of human chorionic gonadotropin/luteinizing hormone receptor gene in brain. Endocrinology, 1993; 132:2262-2270). Furthermore, it is postulated that HCG may play an important signaling role in differentiation and development of tissue subsets from germ cell layering during blastocyst stage (Gallego M J, Porayette P, Kaltcheva M M The Pregnancy Hormones HCG and Progesterone Induce Human Embryonic Stem Cell Proliferation and Diferentiation into Neuroectodermal Rosettes. Stem Cell Research and Therapy 2010; 1:28) to organ development during fetal life (Abdallah M A, Lei Z M, Li x, Greenwold N Human Fetal Non-Gonadal Tissues Contain HCG/LH Receptors. J Clin Endo Metabol 2004; 89:952-56) and perhaps on some more subtle, yet clinically significant way, in adults. Recent evidence confirms the presence of HCG receptors in the adult CNS, and additional evidence supports HCG as a signaling hormone for tissue differentiation and growth. See also Rao C V, Lei Z M The past, present and future of non-gonadal LH/hCG actions in reproductive biology and medicine. Mol Cell Endocrinol. 2007 Apr. 15; 269(1-2):2-8.

Various models point to neuroplasticity effects which HCG, in appropriate dosing, may provide as the facilitator of the clinic benefits the current inventors see in chronic pain patients. Relevant to the pain control phenomenon the current inventors witness, is the work of Meng, Wennert and Chan (Meng X, Rennert O, Chan, W Human chorionic gonadotropin induces neuronal differentiation of PC12 cells through activation of stably expressed lutropin/choriogonadotropin receptor. Endocrinology 2007; 148(12)5765-5873) who conducted a study on neuronal differentiation on PC12 cell lines derived from rat pheochromocytoma. This investigative platform is a well established and accepted theoretical model for study of neuronal differentiation and signaling in humans (Greene L A, Tischler A S Establishment of a noradrenergic clonal line of rat adrenal pheochromocytoma cells which respond to nerve growth factor. Proced Nat Acad Sci 1976. 73:2424-2428).

They showed that stimulation of HCG receptors present on these cells results in visibly appreciated neuroplasticity effects including neuritogenesis and neurite outgrowth—expansion of cell size and establishing new more complex and functional network connections.

Neuroplasticity is a term that denotes the capacity of neurons to alter functionality and, in this case to form new, or perhaps re-establish old, connections. In the context of the aforementioned neuroplasticity changes, HCG has also been shown to stimulate neuronal differentiation of PC12 cells. In the adult brain, although the majority of neurons are post-mitotic, there are multi-potent neural stem/progenitor cells generating young neurons throughout life as noted above (Colcci-D'Amato L, Bonavita V, di Porzio U. The end of the central dogma of neurobiology: stem cells and neurogenesis in adult CNS. 2006 Neurol Sci. 27:266-270). Animal models in support of these concepts demonstrate evidence of neuroplasticity and neuronal renewal stimulated by HCG. Specifically, one group reported that HCG administration in animals with spinal cord injury can significantly improve recovery of motor function (see Patil A A, Nagaraj M P. The effect of human chorionic gonadotropin (HCG) on functional recovery of spinal cord sectioned rats. Acta Neurochir (Wien) 1983 (69):205-218).

Of further interest in the Meng study was that HCG concentrations were maintained in the 200-1000 ng/ml window for these effects to manifest. Earlier studies have shown that the above HCG effects on primary neurons and glial cells were significant only with ambient concentrations of 100-250 ng/ml of HCG. We believe there is a critical window of HCG concentration required to manifest these neuroplasticity effects which may explain the observed clinical activities. Concentrations too high may blunt this effect. This is consistent with other models of HCG action where concentrations in a narrow window were necessary for effect (Maymo J L, Perez A P, Sanchez-Margalet V, Duenas J L. Up-regulation of placental leptin by human chorionic gonadotropin. Endocrinology 2009; 150(1):304-313). This is postulated to be secondary to transient loss of second messenger production (desensitization) and/or loss of cell surface receptors (down regulation) in response to higher doses of the ligand. For example and consisted with this phenomenon, in men treated for hypogonadism with much higher doses of HCG (5000 IU up to 7000 IU/week) the current inventors do not see this pain control phenomenon.

It is entirely conceivable that different concentrations of a drug hormone have different intended and possible effects at different thresholds in the body depending on ambient receptor tissue density characteristics. Decker R, Nygren A, Kristrom B, Nierop A F M, Gustafsson J, Albertsson-Wikland K. Different Thresholds of Tissue-Specific Dose—Responses to Growth Hormone in Short Pre-Pubertal Children. BMC Endocrine Disorders 2012; 12(26).

In a unifying concept, HCG subcutaneous administration within a narrow window of 120 IU/day -170 IU/day will initiate a ‘neuroplasticity effect’ encompassing shifts in ion channel activity, electrical signaling, intracellular molecular signaling and gene transcription resulting in analgesia in those with chronic, central sensitization mediated pain. The analgesic effect of HCG in specifically addressing central sensitization has not previously been identified by the medical community.

Considering the rapidity of responses observed in our retrospective series, the current inventors postulate that the initial and perhaps sustained responses in patients would result from immediate neuroplasticity mediated effects.

An example of one aspect of this type of immediate neuroplasticity effect might include the following: In the suggested model for the ‘central sensitization’ theory of chronic pain, it has been documented that glial cells, in this setting, are a source of maladaptive secretion of inflammatory mediators (nitric oxide, reactive oxygen species, prostaglandins, pro-inflammatory cytokines, nerve growth factor) that serve to facilitate continued release of excitatory neurotransmitters such as Substance P and glutamate which serve to enhance post-synaptic hyperexcitability of pain transmission neurons (PTN) which augments transmission of pain signals resulting in the hyperalgesic and allodynic responses seen with central sensitization phenomenon and chronic pain. Our theory is that HCG might have a beneficial neuroplasticity effect on these glial cells and shift their function away from the release of these mediators to a more normal physiologically functional state associated with dampening or elimination of chronic pain. See also Bradley L A. Pathophysiology of fibromyalgia. Am J Med. 2009 December; 122(12 Suppl) S22.

This represents one example, or part, of what the current inventors theorize is a synergistic pattern of pleiotropic effects that HCG might exert on several levels within the CNS and Peripheral Nervous System (PNS) to restore cellular function and order and thereby reduce pain. Neural damage to either the PNS or CNS provokes a maladaptive neuroplasticity response in nociceptive pathways that drive spontaneous pain and sensory amplification. This maladaptive plasticity leads to persistent changes which, as mentioned earlier, should be considered a true disease state. In the CNS; gene dysregulation, synaptic facilitation, loss of pain inhibition pathways at multiple levels in the neuraxis all work in concert leading to central amplification of pain signaling. See Costgan M, Scholz J, Woolf C J Neuropathic pain: A maladaptive response of the nervous system to damage. Annu Rev Neurosci. 2009; 32:1-32.

Given the multitude of intertwined and interconnected genetic, cellular and molecular components that cause chronic neuropathic pain, targeted pharmacotherapy will most often lead to disappointing results as it is difficult to create a single agent that would modulate this multi-faceted pathophysiology to obtain the desired effect.

On a broader level, the current inventors view that, HCG administration in specific low dosing ranges has the ability to rejuvenate us in certain ways that re-establish normal physiology and function, in this case restoring more functional pain transmission pathways that result in decreased chronic pain. The current inventors postulate, from the above, that it is HCG that plays a principle role in re-setting physiological function at different levels. What better place to look for clinical answers when pathology develops as adults if the machinery—or the blueprint for functionality—is still in place and available? It would just require the appropriate activation, which the current inventors believe they may be observing with these reported cases. HCG carries the innate information, or signaling capacity, to allow for these cellular plasticity effects that can facilitate a return to ‘normal’ homeostasis or healthy neuronal functioning and concomitant diminution or elimination of pain.

Whatever the mechanism may prove to be when elucidated exactly, the current inventors postulate that administration of HCG parenterally in dosing of 120 -170 IU/day is a non-toxic intervention for pain control relative to the toxicity and QOL burdens associated with the pain control interventions our patients required prior to HCG treatment. Compliance with daily injections was 100%.

With regard to fibromyalgia, a complex, poorly understood disorder that has as a central component, chronic neuropathic pain, even the FDA approved medications to treat fibromyalgia pain—two of which are in fact antidepressants—have no effect on depression and anxiety also associated with this disorder. The current inventors have found that HCG, through its pleiotropic, synergistic neuroplasticity effects results in a marked improvement in depressive symptoms for those in our series. The DoloTest™ Spiritual/Social/Sleep construct findings clearly demonstrate this clinical effect and are supported by the information below. This is a remarkable additional benefit for this patient group in particular. See Recla J. New and emerging therapeutic agents for the treatment of fibromyalgia: an update J Pain Res. 2010; 3: 89-103.

It has been noted that Major Depressive Disorder (MDD) and any chronic pain syndrome often present as co-morbid conditions (30-60% of cases in one report) (Bair M J, Wu J, Damush T M, Sutherland J M, Kroenke K. Association of depression and anxiety alone and in combination with chronic musculoskeletal pain in primary care patients. Psychosom Med, 2008: 70(8);890-7).

The present results suggest that HCG acts similarly on neural pathways involved in MDD that are subject to a very similar sensitization or ‘kindling’ phenomenon (implies that each episode of depression make subsequent depressive episodes more likely and less dependent upon an external stimulus such as stress or sickness), where cellular structure and function are modulated and modified through many of the same CNS synaptic, cell signaling and transcriptional pathways, to effect depression. An excellent review on this very topic posits that there may indeed be a shared neurobiological basis of MDD, FM, neuropathic pain, and other chronic pain syndromes which makes the option of HCG treatment an even more intriguing concept as it may, as a single agent, truly address these related and co-morbid CNS disorders concomitantly and with minimal toxicity as compared with current available options (Maletic V., Raison CL Neurobiology of depression, fibromyalgia and neuropathic pain. Frontiers in Bioscience. June 1, 2009; (14):5291-5338). Robert Post first proposed that ‘kindling’ and sensitization may have similar neurobiological underpinnings, such as neuroplastic changes and alterations in gene expression (Post R M. Kindling and sensitization as models for affective episode recurrence, cyclicity, and tolerance phenomenon, Neurosci Biobehav Rev. 2007; 31(6):858-73. Epub 2007 Apr. 24.

Others have reported on the use of subcutaneous HCG for purported pain control but have advocated for higher dosing, far above what the current inventors have used and are recommending. Tennant recommends in this report using 500 IU to 1000 IU given subcutaneously 1-3 times per week and reports pain control. However, the one quantified case referenced still required 1400 mg of morphine for pain control from a baseline of 3500 mg (Tennant F. Human chorionic gonadotropin: Emerging use in pain management. 2010 Published on Internet and in Practical Pain Management, Jun. 1, 2009). Also, these higher doses advocated would run a significant risk of ovarian hyper-stimulation in women who still have their ovaries, it should be noted. One could also surmise that if larger dosing such as this were to have a significant analgesic effect, this would have been observed on a larger scale in the many thousands of patients treated long term for male hypogonadism on similar dosing regimens. To our knowledge, there are no such reports.

We believe that HCG, used in the fashion described here, will prove to be a useful clinical treatment of any chronic pain condition where central sensitization pathways are at work. Whether this is due to an initial peripheral insult that has been perpetuated through damage or injury to PNS or CNS structures, or in those cases where the initial insult is unknown or unclear, but central sensitization pathways have nonetheless been activated. These disorders would include and not limited to: fibromyalgia; osteoarthritis; rheumatoid arthritis; neuropathy and chronic pain resulting from bulging vertebral discs; post operative pain syndrome; vulvodynia; chronic pain resulting from CNS insults such as stroke, spinal cord injury and multiple sclerosis; and PNS lesions or diseases including: direct nerve trauma, toxic and metabolic neuropathies, herpes zoster and AIDS.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context.

Where the specification claims refers to at least one of something selected from the group consisting of A, B, C . . . and N, the text should be interpreted as requiring only one element from the group, not A plus N, or B plus N, etc. Additionally, although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

1. A method of treating a central sensitization disorder in a subject in need thereof, the method comprising orally administering to the subject a therapeutically effective amount of human chorionic gonadotropin, wherein the therapeutically effective amount is a daily dosage of human chorionic gonadotropin in the range of from about 500 IU/day to about 2000 IU/day.

2. A method of treating chronic pain associated with a central sensitization disorder in a subject in need thereof, the method comprising orally administering to the subject a therapeutically effective amount of human chorionic gonadotropin, wherein the therapeutically effective amount is a daily dosage of human chorionic gonadotropin in the range of from about 500 IU/day to about 2000 IU/day.

3. The method of claim 1 or 2, wherein the central sensitization disorder is selected from the group consisting of: fibromyalgia, irritable bowel syndrome, a chronic arthropathy, post herpetic neuralgia, trigeminal neuralgia, an autonomic neuropathy, a metabolic neuropathy, a post-surgical pain syndrome, a spinal nerve compression syndrome, a migraine, arachnoiditis, complex regional pain syndrome, and osteoarthritis.

4. The method of claim 3, wherein the central sensitization disorder is chronic arthropathy.

5. The method of claim 4, wherein the chronic arthropathy is selected from the group consisting of rheumatoid arthritis, arthritic arthropathy, and psoriatic arthritis.

6. The method of claim 3, wherein the central sensitization disorder is post-surgical pain syndrome.

7. The method of claim 6, wherein the post-surgical pain syndrome is a result of a surgery selected from the group consisting of a thoracotomy, a mastectomy, and an amputation.

8. The method of claim 6, wherein the post-surgical pain syndrome is phantom limb pain.

9. The method of claim 1 or 2, wherein the method comprises administering a tablet.

10. The method of claim 9, wherein the method comprises administering an orally dissolving tablet.

11. The method of claim 1 or 2, wherein the therapeutically effective amount is a daily dosage of human chorionic gonadotropin in the range of from about 700 IU/day to about 1700 IU/day.

12. The method of claim 11, wherein the therapeutically effective amount is a daily dosage of human chorionic gonadotropin in the range of from about 1000 IU/day to about 1500 IU/day.

13. The method of claim 12, wherein the therapeutically effective amount is a daily dosage of human chorionic gonadotropin of about 1000 IU/day.

14. The method of claim 12, wherein the therapeutically effective amount is a daily dosage of human chorionic gonadotropin of about 1500 IU/day.
